(19) United States
(12) Patent Application Publication
(10) Pub. No.: US 2008/0274147 A1
(43) Pub. Date: Nov. 6, 2008

(54) Dry Limited Use Cloth

(75) Inventors: James Schaeffer, Charlotte, NC (US); Cliff Bridges, Greensboro, NC (US)

Correspondence Address: VALERIE CALLOWAY;CHIEF INTELLECTUAL PROPERTY COUNSEL, POLYMER GROUP, INC., 9335 HARRIS CORNERS PARKWAY SUITE 300, CHARLOTTE, NC, 28269, US

(73) Assignee: PGI POLYMER, INC., Charlotte, NC (US)

(21) Appl. No.: 11/911,922

(22) PCT Filed: Apr. 20, 2006
(86) PCT No.: PCT/US2006/015038

§ 371 Date: Jan. 11, 2008

Related U.S. Application Data
(60) Provisional application No. 60/673,207, filed on Apr. 20, 2005.

(51) Int. Cl. A61K 8/02; A61Q 19/00

(52) U.S. Cl. 424/401

(57) ABSTRACT

The present invention is directed to a dry cleansing cloth that comprises at least one personal care composition imparted to the cloth, wherein the composition acts as a visual indicator of the compositions depletion with each use, eventually signaling the consumer for the need of a replacement cloth. Accordingly, the cloth of the present invention may be any suitable limited use substrate, including wovens, nonwovens, films, and combinations thereof. Preferably, the limited use cloth comprises a nonwoven substrate, wherein the nonwoven substrates may include, but are not limited to airlaid fabrics, wetlaid fabrics, spunlace fabrics, spunbond fabrics, meltblown fabrics, coform fabrics, entangled spunbond fabrics, and combinations thereof.

### CROSS-REFERENCE TO RELATED APPLICATION

This application claims the benefit of priority Provisional Application No. 60/673,207, filed Apr. 20, 2005, the disclosure of which is incorporated herein by reference.

### TECHNICAL FIELD

The present invention generally relates to dry limited use cloths, and more specifically relates to dry limited use cloths that include at least one personal care composition imparted to the cloth, wherein the composition acts as a visual indicator of the compositions depletion with each use, eventually signaling the consumer for the need of a replacement cloth.

### BACKGROUND OF THE INVENTION

Over the years, the use of disposable substrates in cleaning and cleansing applications has been well practiced. Suitable substrates have included sponges, woven and nonwoven fabrics, and various combinations thereof. Further, such substrates have been impregnated with home care cleaning agents and personal care cleansing compositions, such as surfactants, solvents, and detergents. The resulting cleaning products fabricated from such impregnated substrates have found acceptance with the general public as a convenient and practical means for cleaning hard surfaces, as well as personal care applications, such as facial cleansing cloths.

Disposable personal care articles, such as facial cloths and body cloths, are not only more convenient, but more hygienic. Bar soap is often utilized by multiple individuals and left to sit near or directly on a bathroom sink or bathtub. If the bar soap isn't in a well drained location, the bar soap will start to soften in the pool of water it sits in and harbor germs. In addition, bar soap can dry out and crack over time. The fine cracks are also known to provide an environment for germs to thrive.

Disposable cloths impregnated or coated with a personal care cleansing composition have eliminated the need for bar soap. The majority of dry disposable personal care products are for the most part women's facial cleansing cloths, infant bathing cloths, or forms of wet wipes. Facial cleansing cloths are single use products usually taking the form of puffs, pads, or sheets that lather in the presence of water. Often, one surface of the facial cloth serves to exfoliate the skin, while the opposing softer surface removes exfoliated cells, make up, and other skin impurities. Infant bathing cloths are also single use products that are usually an embossed nonwoven sheet with a mild cleansing composition imparted thereon for easy bathing of newborns and infants. Single use wet wipes are available for a variety of end uses in both personal care and home care applications.

While single use personal care articles are known in the art, a need remains for a dry limited use cloth imparted with a personal care composition suitable for repeat uses. Further, there remains a need for a limited use cloth wherein the personal care composition provides a visual indication of the depleted personal care composition, prompting the consumer to dispose of the used cloth.

### SUMMARY OF THE INVENTION

The present invention is directed to a dry cleansing cloth that comprises at least one personal care composition imparted to the cloth, wherein the composition acts as a visual indicator of the compositions depletion with each use, eventually signaling the consumer for the need of a replacement cloth. Accordingly, the cloth of the present invention may be any suitable limited use substrate, including wovens, nonwovens, films, and combinations thereof. Preferably, the limited use cloth comprises a nonwoven substrate, wherein the nonwoven substrates may include, but are not limited to airlaid fabrics, wetlaid fabrics, spunlace fabrics, spunbond fabrics, meltblown fabrics, coform fabrics, entangled spunbond fabrics, and combinations thereof.

The limited use cloth of the present invention further includes at least one personal care composition, wherein the composition is visibly distinct from the nonwoven fabric. In one embodiment, a pigment is associated with the personal care composition, which is dissimilar compared to the color of the cloth. The personal care composition may be impregnated or printed onto the cloth. In one embodiment, the personal care composition may be imparted to the cloth in the shape of animals or characters so as to appeal to the younger consumer. In addition, the personal care composition may be imparted to the cloth to identify a company brand name, logo, or design and may further utilize pigments associated with a particular company name or logo.

In accordance with the present invention, the limited use cloth is substantially dry. Once wet, the personal care composition imparted in the cloth is activated and released during the application process. Upon further use of the cloth, the pigment associated with the cleansing composition diminishes or the printed on personal care composition may begin to dissipate from the cloth.

In an alternate embodiment, the nonwoven cloth includes at least two layers with one or more personal care compositions is positioned intermediate the two layers. The one or more personal care compositions may be imparted in a linear or linear-like formation, as an array of geometric patterns, in the form of familiar animal shapes or characters, as a company brand name, logo, or design, or a combination thereof. Further, the imparted personal care composition(s) are visible through at least one layer of the cloth and provide a visual indicator as to when the cleansing cloth is in need of replacement.

In yet another embodiment, the nonwoven limited use cloth includes one or more raised portions, wherein the raised portions provide the cloth with three-dimensionality and enhanced aesthetic appeal. The raised portions again may be imparted to the cloth to depict various designs, animals or characters, company brand names or logos, or combinations thereof. In addition, the raised portions may provide aesthetic appeal, as well as functionality, whereby the personal care composition may be releasably associated with one or more imparted raised portions. In such an embodiment, the personal care composition is visibly identifiable in relation to the one or raised portions. As previously mentioned, the personal care composition is diminished with each use to provide a visual indication of when the composition has been completely exhausted.

Further still, the present invention contemplates a limited use cloth to have more than one texture, wherein a first expansive surface may provide an exfoliating surface and the opposing surface may provide a softer cleansing surface for removal of skin impurities. The present invention further contemplates the incorporation of one or more capillaries defined by one or more apertures within the raised portions of the limited use cloth. The apertures can act as a path for delivering the personal care composition to the surface of the outer most skin contacting surface of the raised portions imparted to the cloth.

While the present invention is especially suited for a limited use personal care cloth, the limited use cloth may also find use as a home care limited use cloth. An example of a home care application may include a dishwashing cloth or hard surface cleaning cloth, wherein the composition imparted to the cloth is a dish detergent or other composition, such as an oil, wax, or disinfectant useful for cleaning hard surfaces. Additional non-limiting end uses for the limited use cloth of the present invention include automotive/marine end use applications, wherein a surface protectant may be imparted to the limited use cloth.

Other features and advantages of the present invention will become readily apparent from the following detailed description, the accompanying drawings, and the appended claims.

### BRIEF DESCRIPTION OF THE DRAWINGS

FIG. 1 is a diagrammatic view of a cleansing cloth made in accordance with the present invention;

FIG. 2 is a diagrammatic view of a cleansing cloth made in accordance with the present invention;

FIG. 3 is a diagrammatic view of a cleansing cloth made in accordance with the present invention; and

FIG. 4 is a schematic view of a complete dispensing system in accordance with the teachings of the present invention.

### DETAILED DESCRIPTION

While the present invention is susceptible of embodiment in various forms, there is shown in the drawings and will hereinafter be described a presently preferred embodiment of the invention, with the understanding that the present disclosure is to be considered as an exemplification of the invention, and is not intended to limit the invention to the specific embodiment illustrated.

A wide variety of materials can be used as the limited use dry cloth fabric of the present invention. Particularly suitable fabrics include various nonwoven substrates. Methods of making nonwoven substrates are well known in the art. Non-limiting examples of nonwoven fabrics include carded fabrics, airlaid fabrics, wetlaid fabrics, spunlace fabrics, meltblown fabrics, coform fabrics, and spunbond fabrics.

Non-limiting examples of synthetic materials useful in forming substrates of the present invention include those selected from the group consisting of acetate fibers, acrylic fibers, cellulose ester fibers, polyamide fibers, polyester fibers, polyolefin fibers, such as polyethylene and polypropylene, polyvinyl alcohol fibers, and mixtures thereof. Additional suitable synthetic fibers include, monocomponent fiber, bicomponent fiber, such sheath-core, side-by-side, and islands in the sea, copolymer, elastomers, and combinations thereof, as well as fibers or filaments of various cross sections, such as bi-lobal, tri-lobal, penta-lobal, and other non-round cross sections.

Non-limiting examples of natural materials useful in the present invention include silk fibers, keratin fibers and cellulosic fibers. Nonlimiting examples of keratin fibers include those selected from the group consisting of wool fibers and the like. Nonlimiting examples of cellulosic fibers include those selected from the group consisting of wood pulp fibers, cotton fibers, hemp fibers, jute fibers, flax fibers, and mixtures thereof.

Nonwoven fabrics, regardless of its composition, is subsequently subjected to at least one of several types of bonding operations to bond the individual fibers together to form a self-sustaining web. The present invention may include one or more nonwoven layers prepared by a variety of processes including hydroentanglement. Moreover, the fabric used in the present invention preferably includes a spunbond layer. In addition, a multi-layered cloth may further include films and other non-fibrous materials.

The present invention further contemplates the use of continuous or discontinuous nano-denier filament fabrics, which may be obtained from both splittable fiber and non-splittable fibers. Examples of such suitable fabrics are disclosed in U.S. Pat. No. 6,114,017, entitled, “Micro-Denier Nonwoven Materials Made Using Modular Die Units”, issued Sep. 5, 2000, to inventors Fabbricante, et al., U.S. Pat. No. 6,382,526, entitled, “Process and Apparatus for the Production of Nanofibers”, issued May 7, 2002, to inventors Reneker, et al., and U.S. Pat. No. 6,520,425, entitled, “Process and Apparatus for the Production of Nanofibers”, issued Feb. 18, 2002, to inventor Reneker, all incorporated herein by reference.

One preferred embodiment of the limited use cloth of the present invention includes a 77 grams per square meter (gsm) cloth having a 12 gsm polypropylene spunbond layer and layer including a blend of 18.5 gsm wettable polypropylene staple fiber, 18.5 gsm rayon fiber, and 28 gsm cotton. The two layers were bonded by hydroentanglement and imparted with a plurality of raised portions also by way of hydroentanglement on a foraminous surface. Additionally, the cloth was imparted with a binder to at least on side in order to enhance the scrubbing aesthetic of the cloth.

It's in the purview of the present invention, to further treat the nonwoven fabric with a binder system to improve the release performance of the personal care composition from the limited use cloth. It is believed that certain nonwoven fabrics inherently retain certain chemical compositions within the nonwoven, which affects the amount of soap, cleanser, lotion, or other chemical composition that is released from the fabric. Anionic binder systems, for example, assist with the release of the composition imparted to the fabric.

In accordance with the present invention, the limited use cloth includes one or more personal care compositions that are impregnated, printed, sprayed, coated, or otherwise imparted to the cloth. Personal care compositions may include non-aqueous formulations, such as gels, lotions, film soaps, and powders. One preferred personal care composition is a dry soap. Suitable personal care compositions may further include a variety of ingredients which enhance, protect, or otherwise benefit the skin. Non-limited examples include anionic, nonionic, zwitterionic and amphoteric lathering surfactants, non-lathering surfactants, alpha-hydroxycarboxylic acids and other anti-wrinkle additives, vitamin compounds in either natural or synthetic form, emollients, sunscreen agents, UV protectants, humectants, anti-acne treatments, insect repellents, fragrances, botanicals, antimicrobials, soothing and healing agents, anti-caking agents, antioxidants, and astringents.

Additional suitable personal care compositions are further described in U.S. Pat. No. 4,948,585, entitled, “Washcloth Containing Cleansing Agent”, issued Aug. 14, 1990, to inventor Schlein, U.S. Pat. No. 5,951,991, entitled, “Cleansing Products with Improved Moisturization”, issued Sep. 14, 1999, to inventors Wagner, et. al., U.S. Pat. No. 5,980,931, entitled, “Cleansing Products Having a Substantially Dry Substrate”, issued Nov. 9, 1999, to inventors Fowler, et. al., U.S. Pat. No. 6,063,397, entitled, “Disposable Cleansing Products for Hair and Personal Care”, issued May 16, 2000, to inventors Fowler, et al., U.S. Pat. No. 6,132,746, entitled, “Cleansing Products with Improved Moisturization”, issued Oct. 17, 2000, to inventors Hasenoehrl, et al., U.S. Pat. No. 6,153,208, “Cleansing and Conditioning Article for Personal Care or Hair”, issued Nov. 28, 2000, to inventors McAtee, et al., U.S. Pat. No. 6,280,757, entitled, “Cleansing Articles for Personal Care or Hair”, issued Aug. 28, 2001, to inventors McAtee, et. al., U.S. Pat. No. 6,391,835, entitled, “Alkyl Diol Impregnate Dry Cleansing Wipe”, issued May 21, 2002, to inventors Gott, et al., U.S. Pat. No. 6,491,937, entitled, “Cleansing Wipe Article and Method of Manufacture”, issued Dec. 10, 2004, to inventors Slavtcheff, et al.; U.S. Pat. No. 6,495,151, “Cleansing Articles for Personal Care or Hair”, issued Dec. 17, 2002 to McAtee et al., and U.S. Pat. No. 6,638,527, entitled, “Substantially Dry Cleansing Product of Improved Latherability and Wet Flexibility”, issued Oct. 28, 2003, to inventors Gott, et. al., all of which, are hereby incorporated by reference.

It is further in the purview of the present invention that the personal care composition provides a visual indication of the amount of personal care composition available in the cloth. According to the invention, the limited use cloth is initially a substantially dry product, which is exposed to a water source to activate and release the personal care composition during the process of applying the composition to the skin. The cloth may be used more than once, wherein after each use the personal care composition is diminished until the composition has been completely exhausted. Thereafter, the cloth may be discarded and replaced with a fresh cloth.

In one embodiment, the limited use cloth is printed with one or more personal care cleansing compositions, wherein the cleansing composition is visible due to a pigment associated with the composition, which contrasts with the cloth. The personal care cleansing composition may be a single color or multiple colors that are visibly distinct on or within the cloth. Optionally, the personal care cleansing composition may be a thermally influenced composition, wherein the composition may change color during use depending upon the water temperatures utilized with the cloth.

The personal care composition may be imparted to one side or both sides of the cloth to aesthetically appeal to the wide range of consumers, wherein any variety of animals, popular characters, patterns, company brand names, logos, or abstract designs may be utilized, as well as pigments associated with a particular company or logo. Upon each use of the cloth, the pigment associated with the composition diminishes or the imparted personal care composition may begin to dissipate from the cloth.

In an alternate embodiment, the limited use cloth includes at least two layers, wherein each layer has a first outer expansive surface and a second inner expansive surface with one or more personal care compositions positioned intermediate the two inner expansive surfaces. The one or more personal care compositions may be imparted in a linear or linear-like formation, as an array of geometric patterns, in the form of familiar animals or characters, as a company brand name, logo, or design, or a combination thereof. Further, the imparted personal care composition(s) are visible through at least one layer of the cloth and provide a visual indicator as to when the limited use cloth is in need of replacement. Optionally, the cloth may have more than one associated textures, wherein a first expansive surface may provide an exfoliating surface and the opposing surface may provide a softer cleansing surface for removal of dead skin cells, make up, and other skin impurities.

It is further in the purview of the present invention to mechanically or chemically bond the limited use cloth along the outer most edges of the cloth, as well as within the cloth to form one or more “pillow” regions or areas within the cloth wherein the substrate layers remain essentially unattached. The pillow regions are uninhibited by way of movement during the cleaning process contributing to formation of lather and further provide a region to impart one or more desired personal care compositions. Pillow regions are further described in pending PCT Patent Application No. WO04/073479, entitled, “Cleaning Sheets”, published Sep. 2, 2004, to inventors Wong, et al., hereby incorporated by reference.

In yet another embodiment, the cleansing cloth includes one or more raised portions, wherein the raised portions provide the cloth with three-dimensionality and enhanced aesthetic appeal. The raised portions are imparted to the cloth substrate of the present invention by hydroentanglement on a foraminous support member. Foraminous support members include, but are not limited to foraminous metal shells, perforated screens, and ablated thermoplastic drums, wherein the fibers and/or filaments of the substrate are hydraulically influenced into the respective voids embedded within the foraminous support member. One suitable foraminous support member is illustrated as a three-dimensional image transfer device in U.S. Pat. No. RE38,105, entitled, “Apparatus for Making Nonwoven Fabrics Having Raised Portions”, issued May 6, 2003, to inventors James, et. al., hereby incorporated by reference.

According to the present invention, the raised portions may be imparted to the cloth to depict various designs, patterns, animals or characters, company brand names or logos, or the combinations thereof. In addition, the personal care composition may be releasably associated with one or more imparted raised portions, wherein the cleansing composition is visibly identifiable in relation to the one or more raised portions and released from the raised portions to the skin contacting surfaces of the cleansing cloth. As previously mentioned, the cleansing composition is diminished with each use to provide a visual indication of when the composition has been completely exhausted.

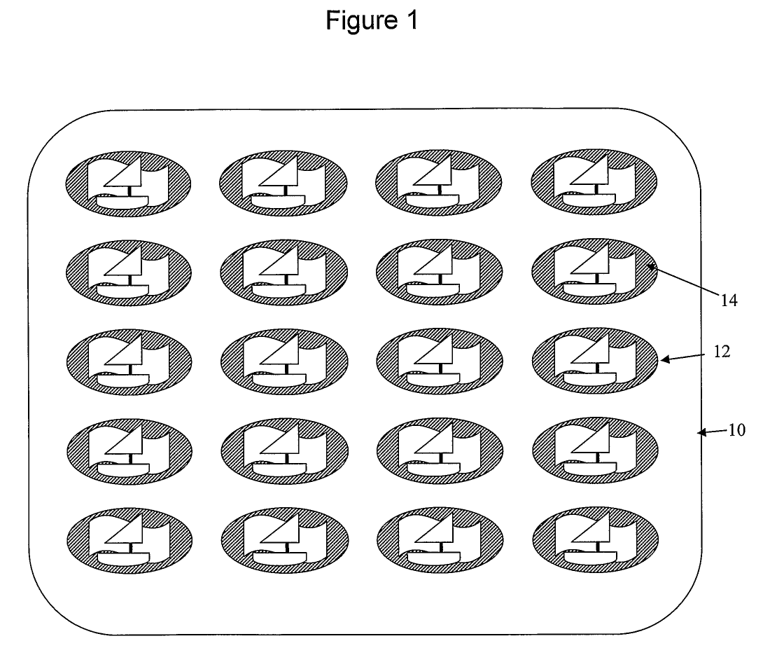

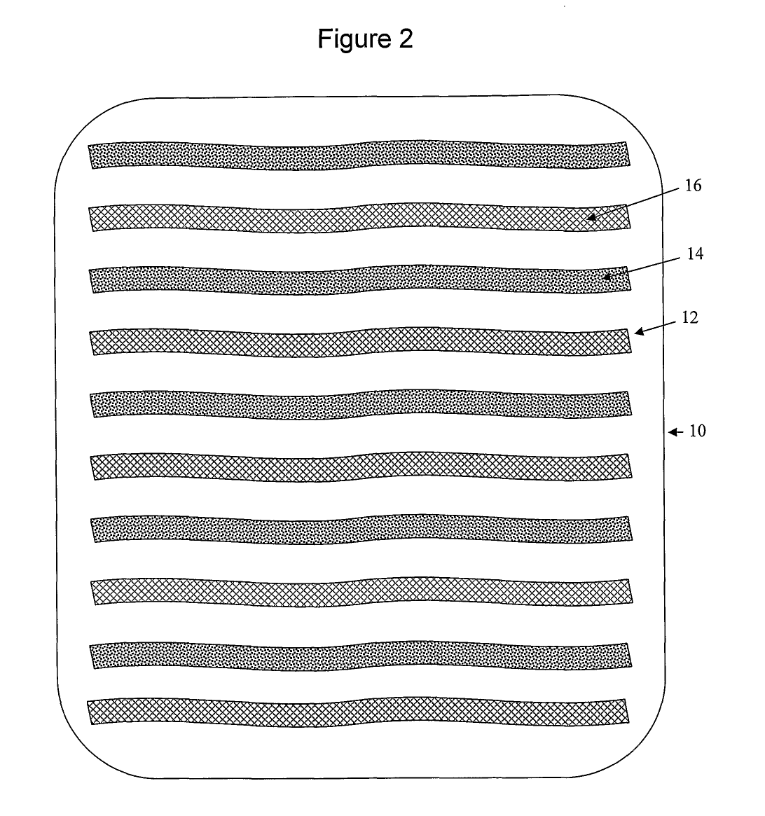

FIGS. 1-3 are diagrammatic views of cleansing cloth embodiments made in accordance of the present invention. FIG. 1 shows cleansing cloth 10 comprising a plurality of designs, which may be printed onto the cloth or imparted as raised surfaces into the cloth 12. When the designs 12 are imparted as raised surfaces, at least a portion of the raised surfaces may be impregnated with a cleansing composition 14. FIG. 2 shows another cleansing cloth 10 comprising a plurality of wave-like designs 12. Such designs may be printed or impregnate with a first personal care composition 14 and a second personal care composition 16.

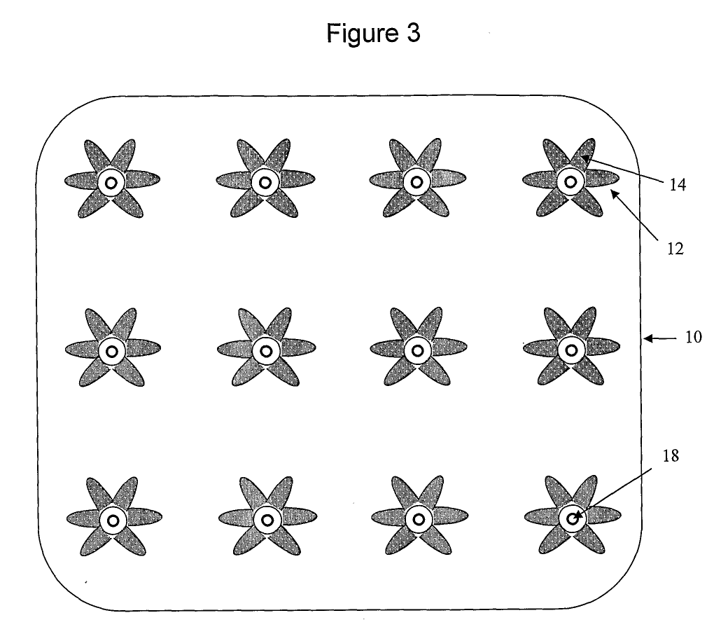

The present invention also contemplates the incorporation of one or more capillaries defined by one or more apertures within the raised portions of the limited use cleansing cloth, as represented in FIG. 3. The apertures 18 can act as a passage for delivering the personal care composition to the surface of the outer most skin contacting surface of the raised portions imparted to the limited use cloth.

The dry limited use cloths are suitable for various personal care applications, such as facial cloths and body cloths for both men and women. In addition to personal care applications, the limited use cloth may also find use as a home care limited use cloth. An example of a home care application may include a dishwashing cloth or hard surface cleaning cloth, wherein the composition imparted to the cloth is a dish detergent or other composition, such as an oil, wax, or disinfectant useful for cleaning hard surfaces. Additional non-limiting end uses for the limited use cloth of the present invention include automotive/marine end use applications, wherein a surface protectant may be imparted to the limited use cloth.

The present invention further contemplates a complete dispensing system, including a plurality of interfolded dry limited use cloths and a dispensing unit that dispenses the cloths one at a time. The dispensing unit may be a disposable unit that is discarded once the supply of cloths has been depleted or alternately, it may be a permanent unit that may be designed to accept a replenished supply of cloths, which may be contained in a film package. When the dispensing unit is a permanent unit, the film packaging may be removed from the unit and discarded each time the limited use cloth supply is depleted.

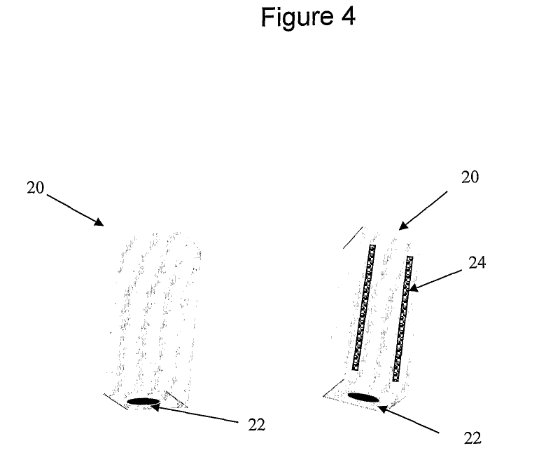

FIG. 4 shows one embodiment of a complete dispensing system 20 made in accordance with the present invention. The dispensing system may be affixed to the showering/bathing area by various means 24, including but not limited to hook and loop fasteners or adhesive tapes. In another embodiment, the complete dispensing system may be designed to hang from or mount to various shower caddy systems. Optionally, the dispensing system may be disposable, wherein the packaging can be readily discarded after the supply of limited use cloths has been depleted. In still another embodiment, the dispensing system may resemble an L-bracket for hanging over the bath tub or shower stall. In addition, the L-bracket may include adhesive means, such as a hook and loop fastener for affixing the dispensing system to the wall or shower stall. The L-bracket dispensing system further includes a means for accepting and securing a supply of limited use cloths in the bracket. Suitable means may include an adhesive strip, hook and loop fasteners, or other means. As illustrated in FIG. 4, the dispensing system 20 may include at least one opening for dispensing a single limited use cloth 22. Optionally, dispensing system may be designed for utilization in close proximity to a kitchen or bathroom sink, wherein the dispensing unit may comprise dry facial cloths, dry cloths for shaving purposes, dry dishwashing cloths, or various other limited use cloths.

From the foregoing, it will be observed that numerous modifications and variations can be affected without departing from the true spirit and scope of the novel concept of the present invention. It is to be understood that no limitation with respect to the specific embodiments illustrated herein is intended or should be inferred. The disclosure is intended to cover, by the appended claims, all such modifications as fall within the scope of the claims.

What is claimed is:

1. A dry limited use cloth activated by a source of water comprising:
a first nonwoven fabric layer having a first expansive surface and an opposing second expansive surface, wherein at least one surface includes at least one raised portion; and
at least one personal care composition imparted to at least one surface, wherein said personal care composition is visibly distinct on said nonwoven fabric and further provides a visible indication of the amount of said composition remaining on said cloth after each use.

2. A dry limited use cloth as in claim 1, wherein said cloth includes a spunbond layer.

3. A dry limited use cloth as in claim 1, wherein said personal care composition is selected from the group consisting of a gels, powders, films, and combinations thereof.

4. A dry limited use cloth as in claim 3, wherein said personal care composition a dry film soap.

5. A dry limited use cloth as in claim 1, wherein said first expansive surface of said nonwoven exhibits a dissimilar texture than said second expansive surface.

6. A dry limited use cloth as in claim 1, wherein said personal care composition is a dissimilar color compared to said nonwoven fabric.

7. A dry limited use cloth as in claim 6, wherein said personal care composition is releasably associated with said raised portions.

8. A dry limited use cloth activated by a source of water comprising:
at least two nonwoven fabric layers, wherein each layer has a first outer expansive surface and a second inner expansive surface, wherein each has at least one raised portion within said outer expansive surface; and
at least one personal care composition positioned between said inner expansive surfaces of said layers, wherein said personal care composition is visibly distinct through at least one layer and provides visual indication of the available amount of composition remaining within said cloth after each use.

9. A dry limited use cloth activated by a source of water comprising:
at least one nonwoven fabric having a plurality of raised portions, wherein said raised portions further include at least one capillary defined by at least one aperture within said raised portion; and
at least one personal care composition, wherein said personal care composition is visibly distinct through said fabric, releasably associated with said aperture(s) positioned within said raised portions, and provides a visual indication of the amount of said composition remaining on said cloth after each use.

10. A complete dispensing system comprising:
a disposable dispensing unit; and
a plurality of inter-folded limited use dry limited use cloths activated by introducing a source of water comprising (i) at least one nonwoven fabric layer having a first expansive surface and an opposing second expansive surface, wherein said nonwoven fabric comprises at least one raised surface; and
at least one personal care composition, wherein said personal care composition provides a visible indication of the amount of said composition remaining on said body cloth after each use.

11. A complete cleansing system comprising:
a dispensing unit; and
a plurality of inter-folded limited use dry cloths activated by introducing a source of water comprising at least one nonwoven fabric layer having a first expansive surface and an opposing second expansive surface, wherein said nonwoven fabric comprises at least one raised surface; and at least one personal care composition, wherein said personal care composition provides a visible indication of the amount of said composition remaining on said body cloth after each use.
